(19) United States
(12) Patent Application Publication
(10) Pub. No.: US 2026/0083832 A1
(43) Pub. Date: Mar. 26, 2026

(54) Vaccine Composition For Stimulation Of Broad Spectrum Memory B Cell Expansion

(71) Applicant: Nant Holdings IP, LLC, Culver City, CA (US)

(72) Inventor: Patrick Soon-Shiong, Culver City, CA (US)

(73) Assignee: Nant Holdings IP, LLC, Culver City, CA (US)

(21) Appl. No.: 19/124,995

(22) PCT Filed: Oct. 10, 2023
(86) PCT No.: PCT/US2023/076431

§ 371 Date: Apr. 28, 2025

Related U.S. Application Data
(60) Provisional application No. 63/381,372, filed on Oct. 28, 2022.

(51) Int. Cl. A61K 39/00; A61K 31/7105; A61K 31/711; A61K 39/21; A61K 39/215; A61K 39/39

(52) U.S. Cl. CPC A61K 39/00114; A61K 31/7105; A61K 31/711; A61K 39/21; A61K 39/215; A61K 39/39; A61K 2039/53; A61K 2039/54; A61K 2039/55527; A61K 2039/575

(57) ABSTRACT

Provided herein are vaccine compositions for use in the stimulation of broad-spectrum memory B cell expansion in a patient that has been exposed or is at risk of exposure to an infectious agent of unknown etiology. The composition comprises IL-15 or an IL-15 analog and at least two toll-like receptor (TLR) agonists, wherein the agonists are targeted to different members of the TLR family. Further contemplated herein are compositions and methods of stimulating germinal B cell expansion in a patient, the composition comprising IL-15 or an IL-15 analog and a nucleic acid, wherein the nucleic acid encodes at least one immunogenic peptide, and wherein the composition is administered either subcutaneously or directly into a lymph node.

### FIELD OF THE INVENTION

The field of the invention is stimulation of B cell expansion for eliciting a comprehensive immune response against various viral and cancer antigens.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The ultimate goal of antiviral vaccines is the eradication of viruses, and virus-infected cells by the immune system. Both the innate and the adaptive arm of the immune system can contribute in such vaccines to eradication of viruses and/or tumor cells, with natural killer (NK) cells and T cells, respectively, as key players. However, vaccines that stimulate and expand the B cells are significantly less explored. B cells are not only producers of antibodies, but also contribute to immune regulation and act as potent antigen-presenting cells. For example, the CD40L/CD40 signaling pathway is the most potent activator of antigen presentation capacity in B lymphocytes, and CD40-activated B cells are potent antigen-presenting cells that induce specific T-cell responses in vitro and in vivo. In preclinical cancer models in mice and dogs, CD40-activated B cell-based cancer immunotherapy was able to induce effective antitumor immunity. Unfortunately, the potential of B cells for cellular therapy in human remains largely unexplored, despite their multiple diverse effector functions.

Upon the SARS-CoV2 disease outbreak, it became abundantly clear that vaccine compositions are needed to fight an infectious agent of unknown etiology. The SARS-CoV2 vaccines took about a year to develop after the outbreak of the disease. But millions of lives could have been saved if there was a vaccine composition that provided temporary protection against an infectious agent of unknown etiology.

Thus, there remains a need in the field to develop vaccine compositions that can be administered to a patient who has been exposed to, or is at risk of exposure to, an infectious agent of unknown etiology.

### SUMMARY OF THE INVENTION

The inventive subject matter is directed to various compositions and methods of immune therapy that produces a comprehensive immune response characterized by significant B-cell activation, expansion, and diversity.

In one aspect of the inventive subject matter, the inventor contemplates a vaccine composition for use in the stimulation of broad-spectrum memory B cell expansion in a patient that has been exposed or is at risk of exposure to an infectious agent of unknown etiology. The composition comprises IL-15 or an IL-15 analog and at least two toll-like receptor (TLR) agonists, wherein the agonists are targeted to different members of the TLR family. In another aspect of the inventive subject matter, the vaccine composition above may be administered to a patient for the purpose of expanding memory B cells in the patient. As should be readily appreciated, the vaccine compositions may be administered to the patient in a single dose, or one or more components may be administered separately (preferably at the same day).

In some embodiments, the agonist is independently selected from the group consisting of a TLR-1 agonist, a TLR-3 agonist, a TLR-4 agonist, a TLR-6 agonist, a TLR-7 agonist, a TLR-8 agonist, a TLR-9 agonist, and a TLR-10 agonist. Preferably, the TLR-4 agonist comprises glucopyranosyl lipid A (GLA), the TLR-7/8 agonist comprises 3M-052, and/or the TLR-9 agonist comprises dsRNA, ssRNA and ssDNA containing unmethylated CpG motifs.

In some embodiments, the IL-15 analog is a pharmacokinetically stabilized IL-15 fusion protein. For example, the IL-15 may be an IL-15/IL-15Rα complex. In a further example, the IL-15 may be a pharmacokinetically stabilized IL-15/IL-15Rα fusion protein complex. In still further examples, the IL-15 is Nogapendekin Alfa Imbakicept (N-803), or a TxM derived therefrom.

It should be appreciated that the inventor also contemplates vaccine compositions for use in expansion of memory B cells in a patient who has been exposed to or is at risk of exposure to an infectious agent of unknown etiology. Therefore, in yet another aspect of the inventive subject matter, the inventor also contemplates a method of stimulating the expansion of memory B cells in a patient, comprising administering to a patient a B-cell vaccine composition comprising IL-15 or an IL-15 analog and at least two toll-like receptor (TLR) agonists, wherein the agonists are targeted to different members of the TLR family.

Also provided herein is a kit for use in stimulating the expansion of memory B cells in a patient. The kit is contemplated to comprise an effective amount of a B-cell vaccine composition comprising IL-15 or an IL-15 analog, at least two toll-like receptor (TLR) agonists, and directions for the use thereof. The agonists are preferably targeted to different members of the TLR family—for example, the kit may contain least two agonists are selected from the group consisting of a TLR-1 agonist, a TLR-3 agonist, a TLR-4 agonist, a TLR-6 agonist, a TLR-7 agonist, a TLR-8 agonist, a TLR-9 agonist, and a TLR-10 agonist. The inventors contemplate the kit to be used by a patient who has been exposed to, or is at risk of exposure to, an infectious agent of unknown etiology.

In yet another aspect of the inventive subject matter, disclosed herein is a vaccine composition for use in the stimulation of germinal B cell expansion in a patient, the composition comprising IL-15 or an IL-15 analog and a nucleic acid. The nucleic acid is contemplated to encode at least one immunogenic peptide. Preferably, the composition is administered to a patient in need of stimulation of geminal B cell expansion either subcutaneously or directly into a lymph node.

In further embodiments, it is contemplated that the immunogenic polypeptide is a viral polypeptide (e.g., coronavirus N-protein, a V3 loop domain of a gp120 HIV protein, or portion thereof) or the immunogenic polypeptide is a tumor associated antigen, a cancer specific antigen, or a tumor- and patient-specific antigen (e.g., tumor associated antigen may be CEA, AFP, EphA3, or CYPB1, and/or wherein cancer specific antigen may be HER2, PSA, PSMA, brachyury, or MUC-1). Therefore, patient may be at risk for cancer or diagnosed with cancer, or the patient may be at risk for a viral infection or infected with a virus.

As will be readily appreciated, it is generally preferred that the IL-15 (IL-15 analog or N803) and immunogenic peptide will be present in an amount effective to stimulate geminal B-cell expansion with specificity against the immunogenic polypeptide, and/or the IL-15 cytokine or cytokine analog will be present in an amount effective to stimulate geminal B-cell expansion with specificity against the immunogenic polypeptide.

Various objects, features, aspects, and advantages of the inventive subject matter will become more apparent from the following detailed description of preferred embodiments.

### DETAILED DESCRIPTION

The inventor has now discovered various compositions and methods for stimulating B cell expansion.

In particular, the inventor found that a combination of multiple polyvalent Toll Like Receptor (TLR) agonists with N803, IL-15, or other IL-15 analog exhibits strong synergy to create a universal B cell vaccine to activate memory B cells. The vaccine disclosed herein provides an initial (or a temporary) protection in response to a pandemic of unknown pathogen and to protect against bioterrorism until the offending antigen is identified. The presently disclosed vaccine also provides an initial protection against tumor associated antigens and/or tumor neoantigens.

The stimulation of broad-spectrum memory B cell expansion elicits a comprehensive, durable, and antigen-specific immune response against one or more antigens that targets a pathogen or cancer antigen-specific antibodies. Thus, the methods presented herein may be used as an initial protection in response to a potential pathogen infection. In addition, it is contemplated that such stimulation will also augment class II presentation of antigens of the pathogen and attendant CD4+ T helper cell activation in the presence of the antigen, thereby enhancing an antigen specific B-cell oriented response. Moreover, it should be appreciated that numerous pathogens are contemplated herein, including protection in response to viral infections, parasite infestations, fungal infections, and mycoplasma infections.

The inventor further contemplates a method for long-term antibody memory and long-term B cell survival with high somatic diversity of B cells. The inventor has surprisingly found that long term antibody memory and B cell survival was observed when the follicular dendritic cell in the germinal center of lymph nodes were presented with IL-15 (or N803) and an antigen stimulus. To that end, the present disclosure provides a vaccine composition comprising IL-15 or N803 or other IL-15 analog together with an immunogenic peptide (for example, HIV envelope immunogen, or any infectious disease antigen, or cancer tumor associated antigen, or cancer neoantigen) administered by directly injecting into lymph nodes to activate follicular dendritic cells, and thereby stimulate and expand germinal B cells having a highly diverse antibody memory.

### Toll Like Receptors (TLR)

Throughout the present disclosure, Toll Like Receptor (TLR) refers in general to any Toll-like receptor of any species of organism. A TLR may be from any mammalian species. TLRs have been identified in various mammalian species including, but not limited to, for example, humans, guinea pigs, and mice. A specific TLR may be identified with additional reference to species of origin (e.g., human, murine, etc.), a particular receptor (e.g., TLR-1, TLR-3, TLR-4, TLR-6, TLR-7, TLR-8, TLR-9, TLR-10), or both.

TLRs are a family of germline-encoded transmembrane proteins that facilitate pathogen recognition and activation of the innate immune system. (Hoffmann J A et al., Science 284, 1313-1318 (1999); Rock F Let al., Proc Natl Acad Sci USA 95:588-593 (1998)). TLRs are pattern recognition receptors (PRRs) and are expressed by cells of the innate immune system, including macrophages, dendritic cells and NK cells.

The present inventor herein discloses novel combinations of TLR agonists with IL-15 or N803 or other IL-15 analogs. The novel combinations of TLR agonists described herein can include two or more, for example three, four, five, six, seven, or eight TLR agonists.

There is evidence that TLR-initiated signals from microorganisms and dead/apoptotic cells within lymphatic tissues contribute to B-cell growth and expansion. The immune system has evolved to recognize selective combinations of these ligands in a synergistic way, in order to induce an appropriate immune response against the infecting microorganism. For example, TLR-3 and TLR-7 synergize with each other in inducing DCs isolated from peripheral blood to produce proinflammatory cytokines and chemokines, and to activate TCR transgenic T cells with appropriate antigen specificity. TLR-3 and TLR-4 each could synergize with TLR-7/8 and 9 to amplify IL-12 production by DCs in vitro, resulting in an enhancement of the Th1 T cell response. Likewise, the triple combination of TLR-3, TLR-4 and TLR-7 has synergistic effects. The inventors have now found that a TxM scaffold or modified IL-15 superagonist (ALT-803) exhibits strong synergy with TLRs in promoting B cell expansion.

In the presently disclosed methods, the TLR agonist is preferably selected from the group consisting of a TLR-1 agonist, a TLR-3 agonist, a TLR-4 agonist, a TLR-6 agonist, a TLR-7 agonist, a TLR-8 agonist, a TLR-9 agonist, and a TLR-10 agonist.

It is contemplated that the TLR-4 agonist comprises glucopyranosyl lipid A (GLA), monophosphoryl lipid A (MPLA) and/or deacylated 3-O-monophosphoryl lipid A (3D-MPLA).

As discussed above, the inventor also contemplates that B-cell stimulation can be effected by inclusion of a TLR-7/TLR-8 agonist into contemplated compositions, and a particularly preferred ligand is 3M-052. Suitable compositions and methods for 3M-052 are described in WO 2012/024284. The TLR-7 and TLR-8 are endosomal receptors that play an important role in the immune response to viral infection. They ordinarily recognize GU-rich short single-stranded RNA (ssRNA) as their natural ligand, as well as small synthetic molecules, such as imidazoquinolines and nucleoside analogs. TLR-7 and TLR-8 signaling is MyD88-dependent and is mediated by the nuclear translocation of AP-1 and NF-κB, in addition to the phosphorylation of interferon regulatory factors (IRFs). IRFs promote the induction of interferon-stimulated response element (ISRE) and the subsequent expression of type I interferon (IFN) genes, whereas AP-1 and NF-κB activation results in the production of pro-inflammatory cytokines. Other ligands are also deemed suitable for use herein and include single-stranded RNAs (e.g., RNA homopolymer ssPolyU and GU-rich oligonucleotides, such as ssRNA40), various base analogs (e.g., imidazoquinoline compounds, such as Resiquimod (R848) and Imiquimod (R837)), the thiazoquinoline compound CL075, and various nucleoside analogs, such as CL264. However, and as noted above, an especially preferred TLR7/TLR8 receptor ligand is 3M-052 that locally stimulates activity without systemic cytokine response (see e.g., Vaccine 2011 Jul. 26;29(33):5434-42).

The TLR-9 agonist contemplated in the present disclosure comprises a CpG oligodeoxynucleotide. For example, the TLR-9 agonist may comprise dsRNA, ssRNA and/or ssDNA containing unmethylated CpG motifs. In some instances, the TLR-9 agonist is an oligonucleotide comprising one or more unmethylated CpG dinucleotide (CpG ODNs). In some instances, the TLR-9 agonist comprises EN101.

### IL-15, N-803, and TxM

The invention is based, at least in part, on the surprising discovery that a TLR agonist in combination with N-803, a complex of an interleukin-15 (IL-15) superagonist mutant and a dimeric IL-15 receptor α/Fc fusion protein, is useful for the stimulating broad spectrum memory B cell expansion in a patient.

Interleukin-15 (IL-15) is an important cytokine for the development, proliferation, and activation of effector NK cells and CD8+ memory T cells. The inventors have now found that that the combination of one or more TLR agonists and IL-15 based molecule (such as N-803 or a TxM) exhibits strong synergy in promoting B-cell growth and proliferation.

To facilitate clinical development of IL-15-based therapeutics, an IL-15 mutant (IL-15N72D) with increased biological activity compared to IL-15 was previously identified by the inventors. The pharmacokinetics and biological activity of this IL-15 super-agonist (IL-15N72D) was further improved by the creation of IL-15N72D:IL-15RαSu/Fc fusion complex (N-803), such that the super agonist complex has at least 25-times the activity of the native cytokine in vivo. Thus, N-803 refers to a complex comprising IL-15N72D noncovalently associated with a dimeric IL-15RαSu/Fc fusion protein and having immune stimulating activity. In one embodiment, the IL-15N72D and/or IL-15RαSu/Fc fusion protein comprises one, two, three, four or more amino acid variations relative to a reference sequence.

In some embodiments, a TxM construct having an IL-15 agonist and receptor portion may be used. Numerous TxM constructs are contemplated. For example, the TxM may be a triple-cytokine fusion molecule, 18/12/TxM, using the N-803 scaffold fused to IL-18 via the IL-15N72D domain and linked to a heteromeric single-chain IL-12 p70 by the sushi domain of the IL-15Rα. This molecule displays trispecific cytokine activity through its binding and signaling through the individual cytokine receptors. In a preferred embodiment, the TxM may be a IL-7/IL-15 TxM as described in US 2021/0290729 A1, the contents of which is incorporated by reference herein.

The inventors therefore contemplate the use of IL-15 and agonist derivatives thereof, wherein the agonist derivatives comprise wild-type and mutant IL-15, IL-15:IL15RaSu, wherein IL-15 is covalently or non-covalently bound to IL-15Ra via the sushi domain (Su), and stabilized fusion proteins derived therefrom. Notably, all variations of IL-15 and agonist derivatives thereof stimulate the IL-15Rbg expressed on immune cells such as T, NK, monocytes, and dendritic cells.

### Vaccine Therapeutics

The present disclosure provides pharmaceutical compositions comprising a TLR agonist and IL-15 (or ALT-803 or other IL-15 analog, or a TxM derived therefrom) for use as a therapeutic. In one aspect, the TLR agonist and IL-15 or its derivative are administered systemically, for example, formulated in a pharmaceutically acceptable buffer such as physiological saline. Preferable routes of administration include, for example, instillation into the bladder, subcutaneous, intravenous, intraperitoneal, intramuscular, or intradermal injections that provide continuous, sustained levels of the composition in the patient. Treatment of human patients or other animals is carried out using a therapeutically effective amount of a therapeutic identified herein in a physiologically acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia or infection. Generally, amounts will be in the range of those used for other agents used in the treatment of other diseases associated with neoplasia or infection, although in certain instances lower amounts will be needed because of the increased specificity of the compound. A compound is administered at a dosage that enhances an immune response of a subject, or that reduces the proliferation, survival, or invasiveness of a neoplastic cell as determined by a method known to one skilled in the art. Alternatively, the compound is administered at a dosage that reduces infection by a virus or other pathogen of interest.

Formulation: The administration of TLR agonist and IL-15 (or ALT-803, or a TxM derived therefrom) for the expansion of broad-spectrum memory B cells may be by any suitable means that results in a concentration of the therapeutic that, combined with other components, is effective in increasing or stabilizing broad spectrum memory B cell growth. TLR agonist and IL-15 (or ALT-803, or a TxM derived therefrom) may be contained in any appropriate amount in any suitable carrier substance and is generally present in an amount of 1-95% by weight of the total weight of the composition. The composition may be provided in a dosage form that is suitable for parenteral (e.g., subcutaneously, intravenously, intramuscularly, intravesicularly or intraperitoneally) administration route. The pharmaceutical compositions may be formulated according to conventional pharmaceutical practice (see, e.g., Remington: The Science and Practice of Pharmacy (20th ed.).

Human dosage amounts can initially be determined by extrapolating from the amount of compound used in mice or nonhuman primates, as a skilled artisan recognizes it is routine in the art to modify the dosage for humans compared to animal models. In certain embodiments it is envisioned that the dosage may vary from between about 0.1 μg compound/kg body weight to about 5000 μg compound/kg body weight; or from about 1 μg/kg body weight to about 4000 μg/kg body weight or from about 10 μg/kg body weight to about 3000 μg/kg body weight. In other embodiments this dose may be about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 μg/kg body weight. In other embodiments, it is envisaged that doses may be in the range of about 0.5 μg compound/kg body weight to about 20 μg compound/kg body weight. In other embodiments the doses may be about 0.5, 1, 3, 6, 10, or 20 mg/kg body weight. Of course, this dosage amount may be adjusted upward or downward, as is routinely done in such treatment protocols, depending on the results of the initial clinical trials and the needs of a particular patient.

In particular embodiments, the TLR agonist and IL-15 (or N-803, or a TxM derived therefrom) are formulated in an excipient suitable for parenteral administration. In particular embodiments, N-803 is administered at 0.5 μg/kg-about 15 μg/kg (e.g., 0.5, 1, 3, 5, 10, or 15 μg/kg).

### Kits

Pharmaceutical compositions comprising TLR agonist and IL-15 (or N-803, or a TxM derived therefrom) may be assembled into kits or pharmaceutical systems for use in stimulating the expansion of memory B cells in a patient. Kits according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the one or more TLR agonists and IL-15 (or N-803, or a TxM derived therefrom).

The composition, methods and kits disclosed herein may be used to administer to a patient identified as in need of stimulating the expansion of memory B cells. Such identification of a patient can be in the judgment of the patient or a health care professional and can be subjective (e.g., opinion) or objective (e.g. measurable by a test or diagnostic method). For example, the composition, methods, and kits disclosed herein may be used by a patient who has been exposed to, or is at risk of exposure to, an infectious agent of unknown etiology.

### Stimulation of Germinal B Cell Expansion

In a further aspect of the present disclosure, the inventors contemplate a vaccine composition for use in the stimulation of germinal B cell expansion in a patient. The composition comprises IL-15 (or N-803 or other IL-15 agonist, or a TxM derived therefrom) and a nucleic acid. It is contemplated that the nucleic acid encodes at least one immunogenic peptide. The composition is preferably administered either subcutaneously or directly into a lymph node of a patient who is in need thereof.

The at least one immunogenic peptide is selected from the group consisting of an infectious agent, a tumor associated antigen, a tumor specific antigen, and a neoantigen. In an especially preferred embodiment, the immunogen is an HIV envelope immunogen. The immunogenic peptide as discussed above may be complexed with a carrier or as a mixture with an adjuvant. The vaccine composition can be administered to the patient by any of a number of suitable routes, the most preferable routes being subcutaneous injection or injection into a lymph node.

In this regard, it should be noted that adaptive immune responses are initiated in secondary lymphoid organs, including the lymph nodes. B cells and T cells are sequestered in distinct regions of the lymph nodes, termed the “B cell zone,” located in the outer cortex of the lymph node, or follicles, and the “T cell zone,” which is more diffusely distributed in the area surrounding the follicles (also known as the paracortex) respectively. B cells and T cells express receptors that allow them to home to these respective zones so that they can be exposed to antigen. Intact antigens are present in the B cell zone, whereas in the T cell zone, antigens are presented by antigen-presenting cells, such as dendritic cells. The inventor made the surprising discovery that the direct administration of an immunogenic peptide along with IL-15 (or N-803, or a TxM derived therefrom) to the lymph nodes leads to stimulation of geminal B cell expansion in the patient.

It should be recognized that the survival and proliferation of antigen-stimulated B cells within the germinal center (GC) are crucial for humoral immune responses with high affinity antibodies against infectious agents. Where the payload is a nucleic acid, such nucleic acid can encode a variety of therapeutically effective molecules, and most preferably molecules can act as an antigen in immune therapy of viral infections and cancer. For example, and without wishing to be bound to the following specific antigens, it is contemplated that where the vaccine compositions are used for antiviral treatment of HIV, the antigen may be a protein or nucleic acid encoding a protein that is highly conserved among various clades of the HIV virus such as the V₃ loop region of the gp120 protein, or the N-protein of a variety of coronaviruses (and especially SARS-CoV). On the other hand, where the vaccine compositions are used in the treatment of a cancer, the antigen may be a protein or nucleic acid encoding a cancer-associated or cancer specific antigen such as CEA-1, MUC1, PSA, PSMA, HER2, etc., or one or more tumor and patient specific neoantigens (typically arranged as a polytope).

The immunogenic peptide disclosed herein may include carcinoembryonic antigen (CEA), which is overexpressed in most colon, rectum, breast, lung, pancreas, and gastrointestinal tract carcinomas, or heregulin receptors (HER-2, neu or c-erbB-2), which are frequently overexpressed in breast, ovarian, colon, lung, prostate, and cervical cancers. Likewise, epidermal growth factor receptor (EGFR) can be used as a binding target for the *bispecific antibodies, which is highly expressed in a range of solid tumors including those of the breast, head and neck, non-small cell lung, and prostate, or the asialoglycoprotein receptor, transferrin receptor, and/or serpin enzyme complex receptor, which are expressed on hepatocytes. Similarly, the fibroblast growth factor receptor (FGFR) may be used as a binding target, which is often overexpressed on pancreatic ductal adenocarcinoma cells, and vascular endothelial growth factor receptor (VEGFR) is commonly found in cancer neovasulature. In still further examples, the folate receptor may serve as a binding target for the bispecific antibody, which is selectively overexpressed in 90% of non-mucinous ovarian carcinomas.

Moreover, and as already noted above, one or more neoantigens expressed by tumor cells and presented at the surface of a cancer cell may serve as a binding target for the bispecific antibodies. Therefore, further exemplary antigens with known association with cancer include CEA, MUC-1, EphA3, and CYPB1, and portions thereof, as well as cancer specific antigens such as Her-2, PSA, brachyury, etc., portions thereof.

### Neoepitopes/Neoantigens

Viral neoepitopes may be acquired by (e.g., retro) viral insertion of DNA into a host genome and attendant change in the genomes of the host and pathogen (e.g., via increased mutation rate or genomic instability after HPV integration) or introduced into a host via targeted integration (e.g., via a CRISPR/Cas9 or CRISPR/Cpf1 cassette) of a recombinant nucleic acid encoding the neoepitope(s). Most preferably, targeted insertion is in or proximal a nucleic acid sequence associated with the pathogen (e.g., virus) or disease (e.g., oncogen). Most typically, the viral neoepitopes are associated with a disease or pathogen, where the neoepitope is either acquired and/or artificially introduced. Methods of obtaining viral neoepitopes are further described in U.S. Pat. No. 10,339,274B2, which is incorporated by reference in its entirety.

The cancer neoantigens or neoepitopes disclosed herein may be obtained as disclosed in U.S. Pat. No. 11,421,016 B2, which is incorporated by reference in its entirety. In one contemplated method, the neoepitopes may be obtained by a method that comprises an in silico analysis portion and an in vitro synthesis portion. Most typically, the in silico analysis commences with an omics analysis to identify mutations in the tumor relative to normal tissue of the same patient, and a step of calculation of one or more neoepitopes is performed to define neoepitopes that are specific to the tumor and patient. So obtained neoepitopes are further subjected to filtering step to, for example, eliminate weakly or non-expressed neoepitopes. Identified expressed neoepitopes may then be subject to additional filtering algorithms, for example, to limit neoepitopes to extracellularly exposed or MHC-I bound neoepitopes.

Furthermore, as described in U.S. Pat. No. 11,361,841B2 (which is incorporated by reference in its entirety), the patient-specific neoantigens/neoepitopes may be filtered for various criteria to confer a functional advantage to the tumor/viral cell.

The whole genome sequences (tumor and matched normal) may be analyzed using the BAMBAM tools as disclosed in U.S. Pat. No. 9,652,587B2, which is incorporated by reference herein. BAMBAM simultaneously analyzes each genomic position from a patient's tumor and germline genomes using the aligned short-read data contained in SAM/BAM-formatted files (SAMtools library; Li H, Handsaker B, Wysoker A, Fennell T, Ruan J, Horner N, Marth G, Abecasis G, Durbin R; 1000 Genome Project Data Processing Subgroup. The Sequence Alignment/Map format and SAMtools. Bioinformatics. 2009 Aug. 15; 25 (16):2078-9. Epub 2009 Jun. 8). The BAMBAM tool may be used as a sequence analysis engine to compare sequences, the sequences comprising strings of information.

Highly accurate alignment of various closely related sequences may be achieved using the methods disclosed in U.S. Pat. No. 11,393,557 B2, which is incorporated by reference in its entirety. In this approach, whole genome sequences or whole chromosome sequences are processed using de Bruijn graph-based methods in conjunction with a reference sequence with known sequence information, and statistical and heuristic analysis. The processing includes decomposing patient sequence reads into a plurality of respective sets of k-mers, and a composite de Bruijn graph is then generated using the reference sequence and the plurality of respective sets of k-mers. Such analysis is particularly advantageous for HLA determination from DNA and/or RNA sequencing information since each HLA-type has numerous often very similar alleles, and as traditional alignment methods typically fail to have significant differentiation capabilities where sequences have high degree of similarity.

Regardless of the particular type of the immunotherapeutic composition, it should be recognized that the determination of treatment relevant neoepitopes can be iteratively done after first and/or successive rounds of treatment (which may include surgery, immunotherapy, radiation, chemotherapy, etc.) as further disclosed in U.S. Pat. No. 10,532,089 B2, which is incorporated by reference in its entirety. Most typically, the so obtained info on the newly identified treatment relevant neoepitopes may be used in further immunotherapy (e.g., recombinant adenovirus, or synthetic antibodies, possibly in combination with modified NK cells, all optionally in conjunction with checkpoint inhibitors). Moreover, it is generally contemplated that where immunotherapy is employed, conventional chemotherapy treatment may be performed at a relatively low dose to support or maintain immune function. For example, chemotherapy may be performed using a low-dose regime (e.g., between 0.1% and 1%, or 1% and 5%, or 5% and 10%, or 10% and 20%, or higher but less than 50%, or less than 60% or less than 75% of conventional (see prescribing info) dosage).

Multiple neoepitopes may be combined to form a rational-designed polypeptide with a trafficking signal to increase antigen processing and presentation and to so enhance therapeutic efficacy as further disclosed in U.S. Pat. No. 11,779,637, which is incorporated by reference in its entirety. Furthermore, the neoepitope vaccine compositions disclosed herein may include prime/boost administration with concurrent and overlapping immune stimulation and checkpoint inhibition regimes as further disclosed in U.S. Pat. No. 11,590,217, which is incorporated by reference in its entirety.

It should be appreciated that the immune therapeutic compositions presented herein will not only have significant and broad-spectrum activity as a vaccine but may also provide functionalities that reduce or reverse immune suppression.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other), and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A vaccine composition for use in the stimulation of broad-spectrum memory B cell expansion in a patient that has been exposed or is at risk of exposure to an infectious agent of unknown etiology, the composition comprising IL-15 or an IL-15 analog and at least two toll-like receptor (TLR) agonists, wherein the agonists are targeted to different members of the TLR family.

2. The composition of claim 1, wherein the TLR agonists are independently selected from the group consisting of a TLR-1 agonist, a TLR-3 agonist, a TLR-4 agonist, a TLR-6 agonist, a TLR-7 agonist, a TLR-8 agonist, a TLR-9 agonist, and a TLR-10 agonist.

3. The composition of claim 1, wherein the IL-15 analog is a pharmacokinetically stabilized IL-15 fusion protein.

4. The composition of claim 1, wherein the IL-15 analog is an IL-15/IL-15Rα complex.

5. The composition of claim 1, wherein the IL-15 analog is a pharmacokinetically stabilized IL-15/IL-15Rα fusion protein complex.

6. The composition of claim 1, wherein the IL-15 analog is Nogapendekin Alfa Imbakicept (N-803), or a TxM derived therefrom.

7. The composition of claim 2, wherein the TLR-4 agonist comprises glucopyranosyl lipid A (GLA).

8. The composition of claim 2, wherein the TLR-7/8 agonist comprises 3M-052.

9. The composition of claim 2, wherein the TLR-9 agonist comprises dsRNA, ssRNA and ssDNA containing unmethylated CpG motifs.

10. A method of stimulating the expansion of memory B cells in a patient, the method comprising administering to a patient a B-cell vaccine composition comprising IL-15 or an IL-15 analog and at least two toll-like receptor (TLR) agonists, wherein the agonists are targeted to different members of the TLR family, and wherein the patient has been exposed to, or is at risk of exposure to, an infectious agent of unknown etiology.

11. The method of claim 10, wherein the at least two agonists are independently selected from the group consisting of a TLR-1 agonist, a TLR-3 agonist, a TLR-4 agonist, a TLR-6 agonist, a TLR-7 agonist, a TLR-8 agonist, a TLR-9 agonist, and a TLR-10 agonist.

12. The method of claim 10, wherein the IL-15 analog is a pharmacokinetically stabilized IL-15 fusion protein.

13. The method of claim 10, wherein the IL-15 analog is an IL-15/IL-15Rα complex.

14. The method of claim 10, wherein the IL-15 analog is a pharmacokinetically stabilized IL-15/IL-15Rα fusion protein complex.

15. The method of claim 10, wherein the IL-15 analog is Nogapendekin Alfa Imbakicept (N-803), or a TxM derived therefrom.

16. The method of claim 11, wherein the TLR-4 agonist comprises glucopyranosyl lipid A (GLA).

17. The method of claim 11, wherein the TLR-7/8 agonist comprises 3M-052.

18. The method of claim 11, wherein the TLR-9 agonist comprises dsRNA, ssRNA and ssDNA containing unmethylated CpG motifs.

19. A kit for use in stimulating the expansion of memory B cells in a patient, the kit comprising an effective amount of a B-cell vaccine composition comprising IL-15 or an IL-15 analog, at least two toll-like receptor (TLR) agonists, and directions for the use thereof, wherein the agonists are targeted to different members of the TLR family, and wherein the patient has been exposed to, or is at risk of exposure to, an infectious agent of unknown etiology.

20. The kit of claim 19, wherein the at least two agonists are independently selected from the group consisting of a TLR-1 agonist, a TLR-3 agonist, a TLR-4 agonist, a TLR-6 agonist, a TLR-7 agonist, a TLR-8 agonist, a TLR-9 agonist, and a TLR-10 agonist.

21. A vaccine composition for use in the stimulation of germinal B cell expansion in a patient, the composition comprising IL-15 or an IL-15 analog and a nucleic acid, wherein the nucleic acid encodes at least one immunogenic peptide, and wherein the composition is administered either subcutaneously or directly into a lymph node.

22. The composition of claim 21, wherein the IL-15 analog is a pharmacokinetically stabilized IL-15 fusion protein.

23. The composition of claim 21, wherein the IL-15 analog is an IL-15/IL-15Ra complex.

24. The composition of claim 21, wherein the IL-15 analog is a pharmacokinetically stabilized IL-15/IL-15Ra fusion protein complex.

25. The composition of claim 21, wherein the IL-15 analog is Nogapendekin Alfa Imbakicept (N-803), or a TxM derived therefrom.

26. The composition of claim 21, wherein the nucleic acid comprises mRNA, a viral vector, a bacterial vector, or a yeast vector.

27. The composition of claim 21, wherein the at least one immunogenic peptide is selected from the group consisting of an infectious agent, a tumor associated antigen, a tumor specific antigen, and a neoantigen.

28. The composition of claim 27, wherein the infectious agent is a virus.

29. The composition of claim 28, wherein the virus is HIV.

30. The composition of claim 28, wherein the virus is a coronavirus.

31. The composition of claim 21, further comprising at least two toll-like receptor (TLR) agonists, wherein the agonists are targeted to different members of the TLR family.

32. The composition of claim 31, wherein the TLR agonists are independently selected from the group consisting of a TLR-1 agonist, a TLR-3 agonist, a TLR-4 agonist, a TLR-6 agonist, a TLR-7 agonist, a TLR-8 agonist, a TLR-9 agonist, and a TLR-10 agonist.
